(19) United States
(12) Patent Application Publication
(10) Pub. No.: US 2013/0177507 A1
(43) Pub. Date: Jul. 11, 2013

(54) Quick Destruction of the Streptococcus Mutans that is Responsible for Tooth Decay

(71) Applicant: José Luis Córdova Rojas, Santiago (CL)

(72) Inventor: José Luis Córdova Rojas, Santiago (CL)

(21) Appl. No.: 13/734,935

(22) Filed: Jan. 5, 2013

Related U.S. Application Data
(60) Provisional application No. 61/584,368, filed on Jan. 9, 2012.

(51) Int. Cl. C07D 241/04

(52) U.S. Cl. CPC C07D 241/04
USPC 424/54; 544/399

(57) ABSTRACT

An improved special molecule provides a useful composition with following formula:

R1 R2 . . . C . . .

wherein:

| | |
|---|---|
| R1 is a phenyl hydrazine PM | 108.14 |
| C is a Leucine | 131.17 |
| R2 is a methyl piperazina 111 | 100.16 |
| | 339.69. |

The user friendly molecule inhibits Streptococcus mutans bacteria responsible for tooth decay and is especially useful in products used in oral hygiene.

### FIELD OF THE INVENTION

90% of tooth decay is caused mainly by the S mutans, a bacteria that under poor oral hygiene is capable of invading the teeth, causing cavities that in turn can result in irreparable dental losses. Therefore, it is desired to identify a quick action bactericide molecule (less than 60 seconds) to be included in different products that are used in oral hygiene. The main reason why the molecule must quickly kill the bacteria is because all oral hygiene products remain in the mouth for less than 60 seconds. With this condition it is possible to greatly reduced the number of viable bacteria in the teeth surface, thus also reducing the likelihood of cavities.

### BACKGROUND OF THE INVENTION

The Streptococcus mutans bacteria is that main bacteria that causes tooth cavities in humans.

General characteristics: they are spherical or ovoid shaped of 0.8-1 mm in diameter and are grouped in chains when cultured in liquid medium. They are Gram positive, facultative anaerobic, stationary; some species have a capsule and are not sporulated.

Serological Group: identification criteria for S mutans, through the biochemical and serological tests, which distinguish biotype I or serotype C (c, e, f) of S mutans and biotype IV or serotype d (d, g, h) of S sobrinus, which were the most prevalent biotypes and serotypes. These results coincide with other articles on studies made in humans where the serotype c is the most isolated one, with some variations in the studied populations. Therefore, serotype c has a high prevalence, and has therefore a greater possibility of transmission through the population saliva. For this reason, it should be useful in dental cavities control in humans, through the application of active or passive immunization techniques. Also, the antigenic structure of the S mutans has been stable in time, just as it has been demonstrated by Linossier et al.

Habitat: Mucous membranes of animals and human (including intestinal, genital and respiratory tracts).

Virulence Factors: Cell wall polysaccharides, which determine the Lancefield serological group. Antigen M present is some Streptococcus pyogenes strains has antiphagocytic activity. Besides Streptolysin O and S (hemolysins).

Diseases in Humans: are the main agents responsible for cavities in the dental enamel. In children, the colonization and infection by Streptococcus mutans is a key factor in the risk of developing cavities particularly in developing countries, where these problems have a high prevalence. The Streptococcus mutans microorganisms group have a predominant role in the initiation of enamel cavities that affect the teeth, as well as the cavities that affect the adult tooth root surface, due to its aciduric and acidogenic properties and because it produces extra and intra-cellular polysaccharides.

### Protective Metabolite

Marine bacteria have been often considered as producers of anti-bacterial substances thus allowing to keep the ecological stability of the multiple marine ecosystems, as well as the inter-relationships between micro-organisms in epiphytic environments (Fredrickson y Stephanopoulus, 1981, Lemos et al, 1985, Fabregas et al, 1991). In this context, marine micro-organisms, macroalgae and some invertebrates have been described as producers of biological active metabolites (Stierle et al, 1988). For this reason, the sub-marine world is considered as a huge source of bioactive substances, within which are the antibacterial agents. The action and competence mechanisms between microorganisms are varied, and include the production of antibiotics, bacteriocins, siderophores, lysosomes, protease and even altering the pH through the production of organic acids (Hamdan et al, 1991 and Frey et al, 1996).

The first studies on marine bacteria producers of antibacterial agents were made by Rosenfeld and Zobell (1947). From then on, the search and isolation within marine ecosystems of native bacteria with antagonistic activity over marine and terrestrial pathogens microorganisms, have been made in different habitats such as sea water, sediments, phytoplancton, vertebrates and invertebrates (Gauthier et al, 1976, Toranzo et al, 1982, Lodeiros et al, 1988, Austin et al, 1995, Riquelme et al, 1996, 1997). These antagonistic interactions of the “bacteria-to-bacteria” type that involve growth inhibition, correspond to a mechanism that can help to keep the composition of bacterial species on a micro-scale level (Long and Azam, 2011), either by competition for nutrients, space, light and/or through the production of various secondary metabolites, such as antibacterial substances (Fredrickson and Stephanopoulus, 1981, Dopazo et al, 1988, Lemos et al, 1991)

There are a number of studies that have examined the frequency of inhibitory interactions of marine origin isolated microorganisms; the majority of them have referred to researches that evaluate the effect of these bacteria against collections of pathogenic fish and shellfish bacterial, as well as in non-marine pathogenic strains in humans (Lodeiros et al, 1989, Fabregas et al, 1991, Riquelme et al, 1996, Leon and Garcia-Tello, 1998).

The pathogenic microorganisms are more and more resistant to a multiplicity of drugs and this is of great concern for the World Health Organization. It is imperative, therefore, to find new sources of bioactive natural products to counteract the negative effects of these agents in the coming decades and the sea is demonstrating to be this new source. Marine ecosystems of the North and South of the country fit into this approach, even more, considering that the chemistry of marine organisms has been focused mainly in the study of low latitude species. The study of unique marine ecosystems can lead to the discovery of unusual metabolites.

It has been demonstrated that marine organisms are a rich source of natural bioactive products. For the last 20 years chemists that work in marine natural products in collaboration with pharmacologists, in universities as well as in private enterprises, have describe a rising number of new compounds with interesting bio-medical properties. The number and quality of the seeded generated compounds really justifies marine pharmacological research (Faulkner, 2000). In general, the biological material that has been subject to a more focused research is the marine invertebrate groups, sponges, molluscs, bryozoans, corals and sea squirts. Marine fungi are a group of microorganisms capable of biosynthesize metabolites with novel structures (Atlas and Bortha, 2002). Microbial populations, en general, produce compounds that are not favourable to the growth of other populations. Metabolites naturally produced by these populations could provide benefits in disease control in aquaculture in both human and animal diseases (Lodeiros et al, 1989, Riquelme et al, 1997, Castillo et al, 2000, 2001); in this context, tropical fungi are being incorporated to pre-selection programs as potential drugs producers with new modes of action (De la Rosa and Gamboa, 2003).

Studies have revealed that there is a distribution of marine natural products destined to the production of metabolites, of which there are three phylum that are important to mention: Phylum Poriferae (sponges) make up for a 38%, followed by the Phylum Cnidaria (coelenterata) with a 21% and, finally microorganisms and phytoplancton with a 15% (Blunt et al, 2003).

We are before a potentially useful compounds source, given its peculiarities and its unusual chemical structures; very different from those of land-based metabolites restricted to a known type of structural series, many of them present in the bibliography. It is also necessary to add that marine organisms have no cellular immune system but a chemical one to protect themselves in a dynamic aqueous environment (Jaspars, 1998).

### BRIEF DESCRIPTION OF THE DRAWINGS

FIG. 1: a) Streptococcus mutans colonies in agar TYCSB, b) Liquid culture medium TYCSB.

FIG. 2: Positive extracts that produce mortality in Streptococcus mutans.

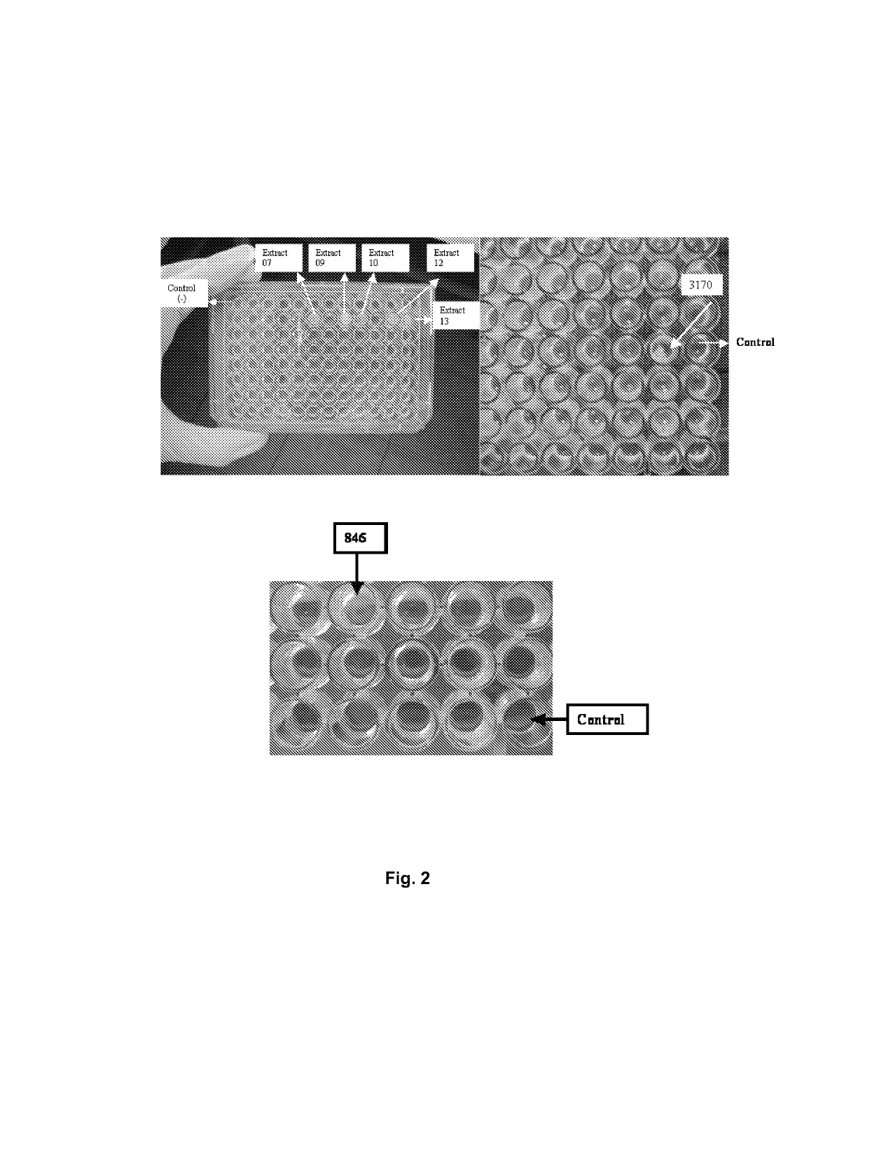

FIG. 3: Positive extracts that produce mortality in Streptococcus mutans plates in agar TYCSB.

FIG. 4: Scheme of catalyzed reactions by drug-metabolizing enzymes, Phase I and Phase II and their impact at a molecular and cellular level.

FIG. 5: Ames test scheme.

FIG. 6: Micronucleusis formation due to the loss of an entire chromosome and acrocentric-type chromosomal fragments.

FIG. 7: Estimation of genetic instability by micronucleus testing.

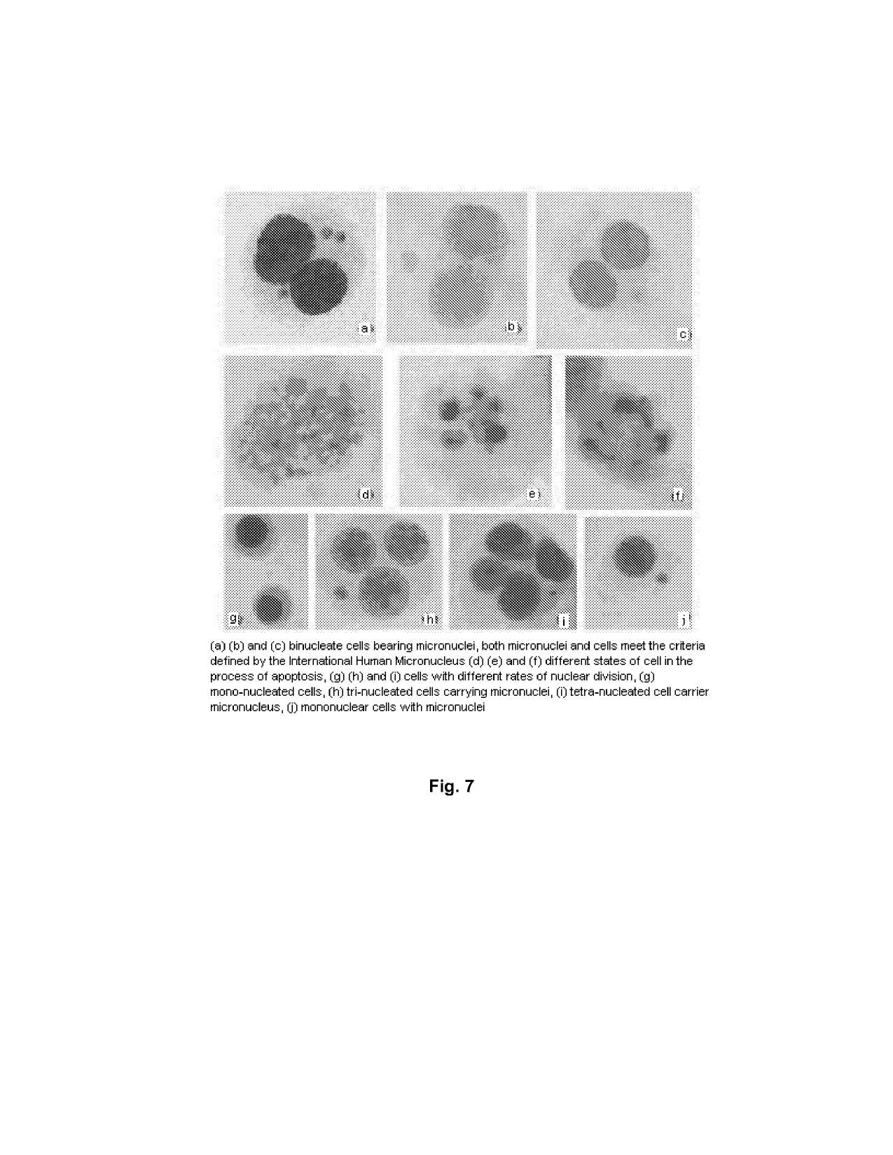

FIG. 8: Microscopic observation of anaphase.

### DETAILED DESCRIPTION OF THE INVENTION

The following is a detailed description and explanation of the preferred embodiments of the invention and best modes for practicing the invention.

Material and Method

The Streptococcus mutans ATCC S-276 strain was obtained from a lamb blood agar plate and propagated in laboratory in a solid and liquid mediums (TYCSB agar and TYCSB selective culture medium) supplementing it with Bacitracin that provides the selectivity required the isolation of the Streptococcus mutans. These bacteria were incubated using an anaerobiosis system (Gas-Pack jars) with a mixture of 95% N₂, 5% of CO₂ during 48 hours at 37° Celsius.

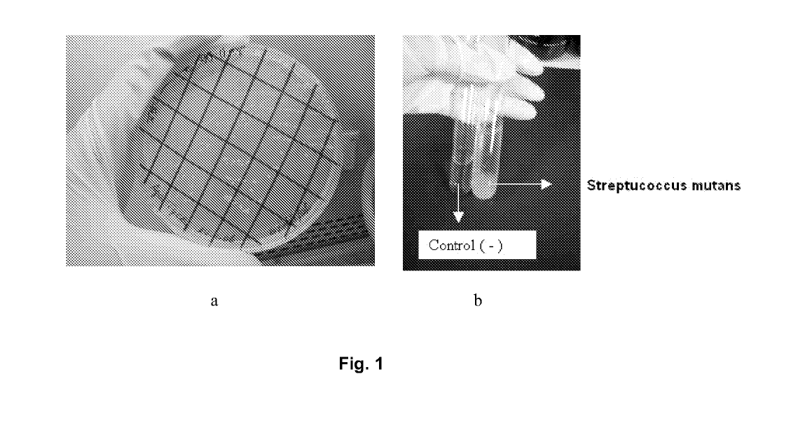

Once the bacteria grew, it was possible to view with the naked eye white colonies from which was extracted one colony (separately) for Gram staining and to confirm that it was a Gram positive focus and. On the other hand, it was grown in a liquid medium so as to be able to detect the Streptococcus mutans growth inhibitors (FIG. 1).

The bacterium was preserved and stored in a 20% glycerol culture medium.

Test Inhibitors (Metabolites)

a) Liquid Medium

Different microorganisms extracts were tested on Streptococcus mutans liquid cultures media, to determine which one of them inhibited or produced mortality in the bacteria. ELISA plates were used that had their wells filled with 50 μl of bacterial culture medium plus 15 μl of extract, thus giving immediate mortality rates (Table 1).

TABLE 1
Extracts Effectiveness on Streptococcus mutans mortality:

| Extract No. | Mortality Rate |
|---|---|
| 846 | Aprox. 10 min. |
| 3170 | Aprox. 10 min. |
| 7 | Instantaneous |
| 9 | Instantaneous |
| 10 | Instantaneous |
| 12 | Instantaneous |
| 13 | Instantaneous |

b) Solid Medium

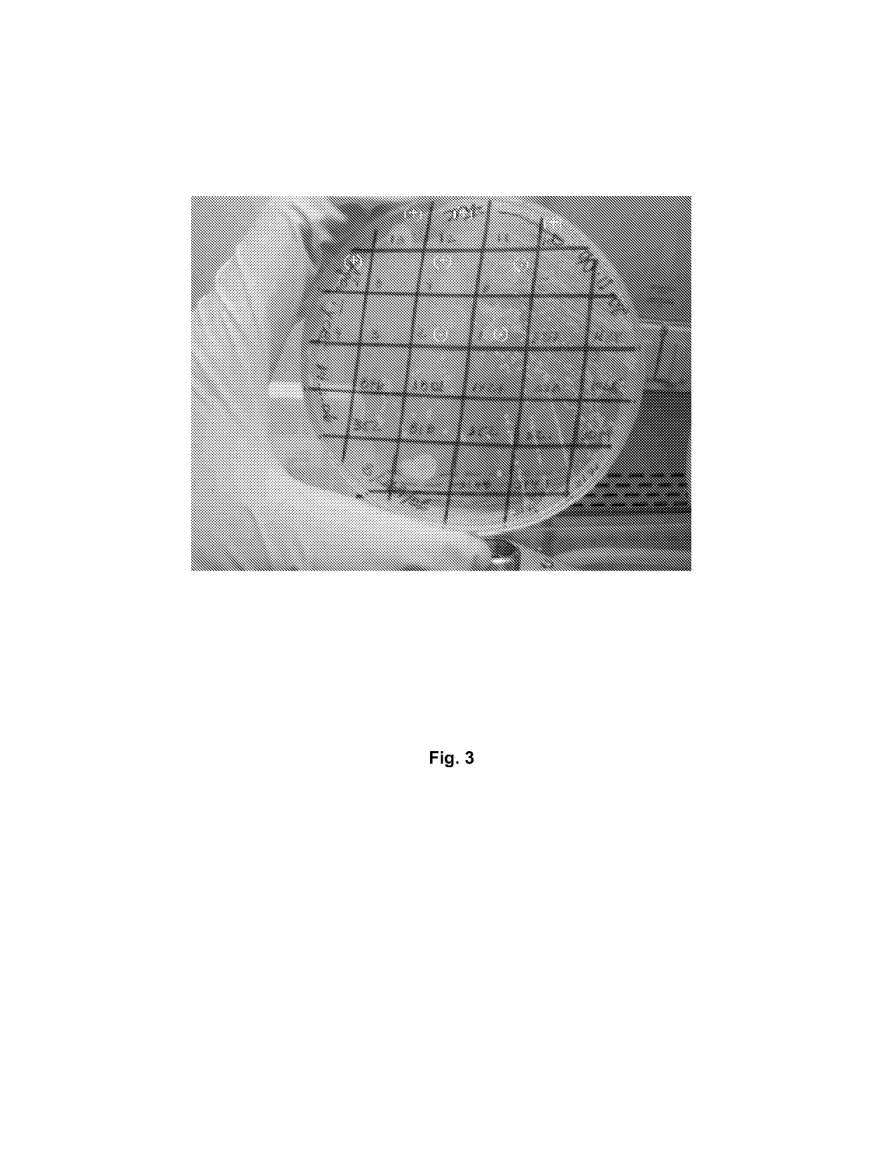

Extracts were tested on the Streptococcus mutans colonies in agar TYCSB giving the same results that in a liquid medium, except for extracts Nos. 1, 2 and 6, which produced a slight white stain but did not killed the bacteria (FIG. 3).

### Extraction and Purification of the Metabolite to Mass Produce It

To isolate and purify the compound(s) present in each one of the extract with inhibitory activity, it was proceeded as follows:

It was studied the most suitable organic medium to re-suspend in the adequate organic solvent so as to maintain the physics-chemical properties (LogP, polarity index, dielectric constant, etc) similar to the solvent used in the initial extraction, in order to corroborate the effectiveness of the original extract.

Then, a thin-layer chromatography (TLC) was performed, studying the most suitable mobile phase, using solvents with appropriate physics-chemical characteristics (particularly, polarity index, polarity and dielectric constants, which are fundamental in liquid chromatography) for optimal separation of the molecules present in the extract.

Subsequently, and with the data obtained from the thin-layer chromatography (especially in the mobile phase), it was performed a column chromatography (CC), under the same conditions in which were the thin-layer chromatography performed (TLC), and this allowed the obtention of fractions with greater amounts of each of the separate molecules.

Each one of the fractions obtained in column chromatography (CC) was analyzed by using high-performance liquid chromatography (HPLC), to corroborate the purity of the previously obtained fractions.

With certainty of the purified fractions, there were some biological tests conducted with each one of them, so as to identify the molecule(s) that were responsible of the S. mutans inhibiting activity.

Confirmation of the Purified Metabolite Efficiency

To identify in the identified extracts, the principle active(s) responsible from the anti-S. Mutans activity from the natural origin extracts, it was proceeded in the following manner:

Once separated and identified each of the extract compounds, there were submitted to a biological evaluation, to prove which one presented the anti-S. Mutans activity.

Each one of the isolated and identified products, were separately compared with the crude extract as a control.

Determination of the Metabolite Structure

To structurally identify the compound(s) that is(were) present in each of the extracts, it was proceeded in the following manner:

Each of the present products (stain/peak) obtained from the TLC, CC and HPLC, in an isolated way, were submitted to infrared (IR) analysis studies, elemental analysis (% percentage of carbon, nitrogen, hydrogen and sulphur (CHNS), proton and carbon magnetic resonance (1H-RMN and 13C-RMN) and mass analysis.

When studying the result of the analysis, it was proceeded to the structural elucidation of the compound(s) that is(were) present.

It was corroborated through a bioinformatic study the chemical validity of the proposed structure(s).

Purification or Synthesis of the Blocking Metabolite.

If there is the possibility of extracting it in an efficient manner, the metabolite must be purified. If that is not possible, the metabolite should be synthesized. The metabolite synthesis protocol should be defined once the problem of not being able to extract it, is confronted.

For the second stage, the genotoxic metabolite features, the following methodology was used:

Bactericidal Genotoxic Metabolite Analysis

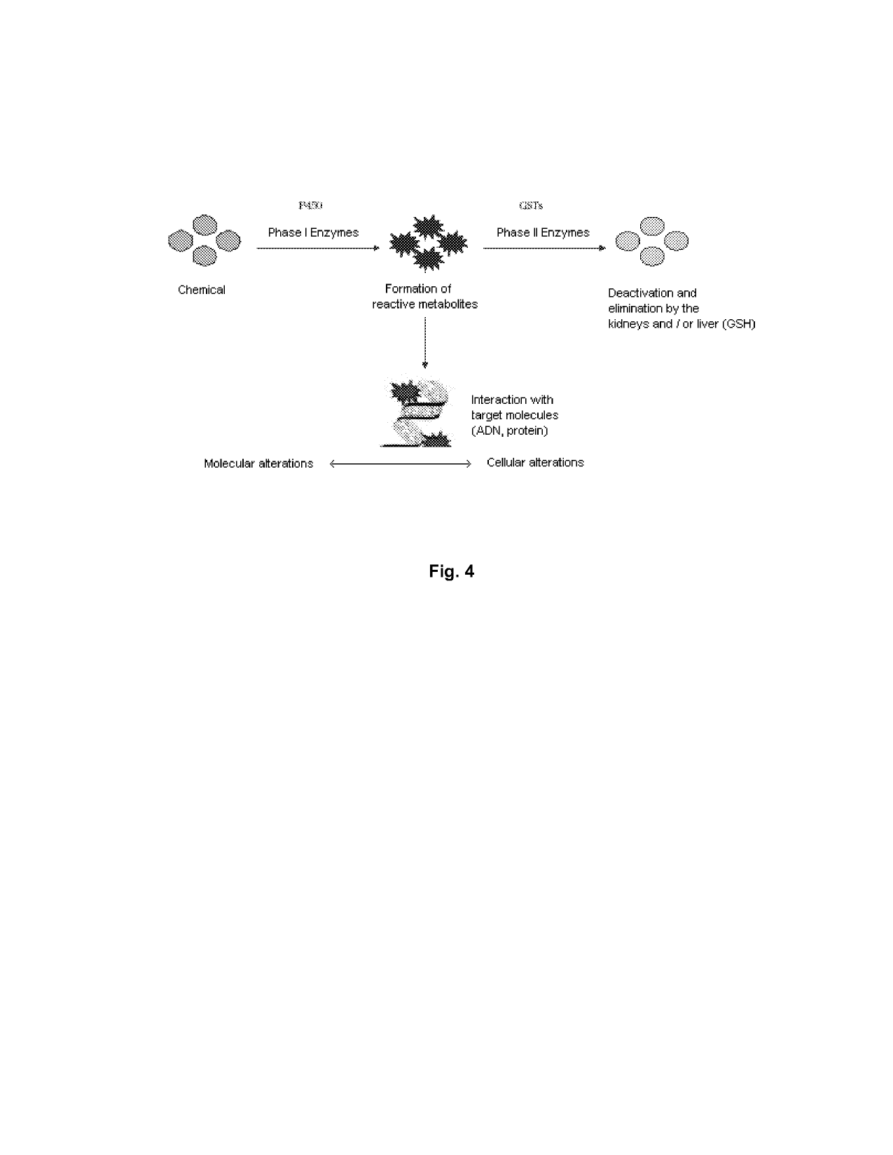

A product with a specific biological activity to be used in animals and/or humans should be analyzed in several stages that go from a determination in vitro of the products to the in vivo analysis. All of the stages are necessary to be able to ensure that this product does not have a direct or indirect side effect that could be harmful to the body (FIG. 4).

Once the structural and functional analysis is performed, toxicogenetic analysis studies were made and a test in an animal model to study the product efficiency.

The toxicogenetic evaluation that was made in vitro and in vivo is used for different biological products.

In the in vitro analysis were conducted the known Ames Test, Induct Test, the anaphase aberration test and of sister chromatid exchange. The analysis of this product using these techniques allowed us to determine the absence of non-desired secondary effects.

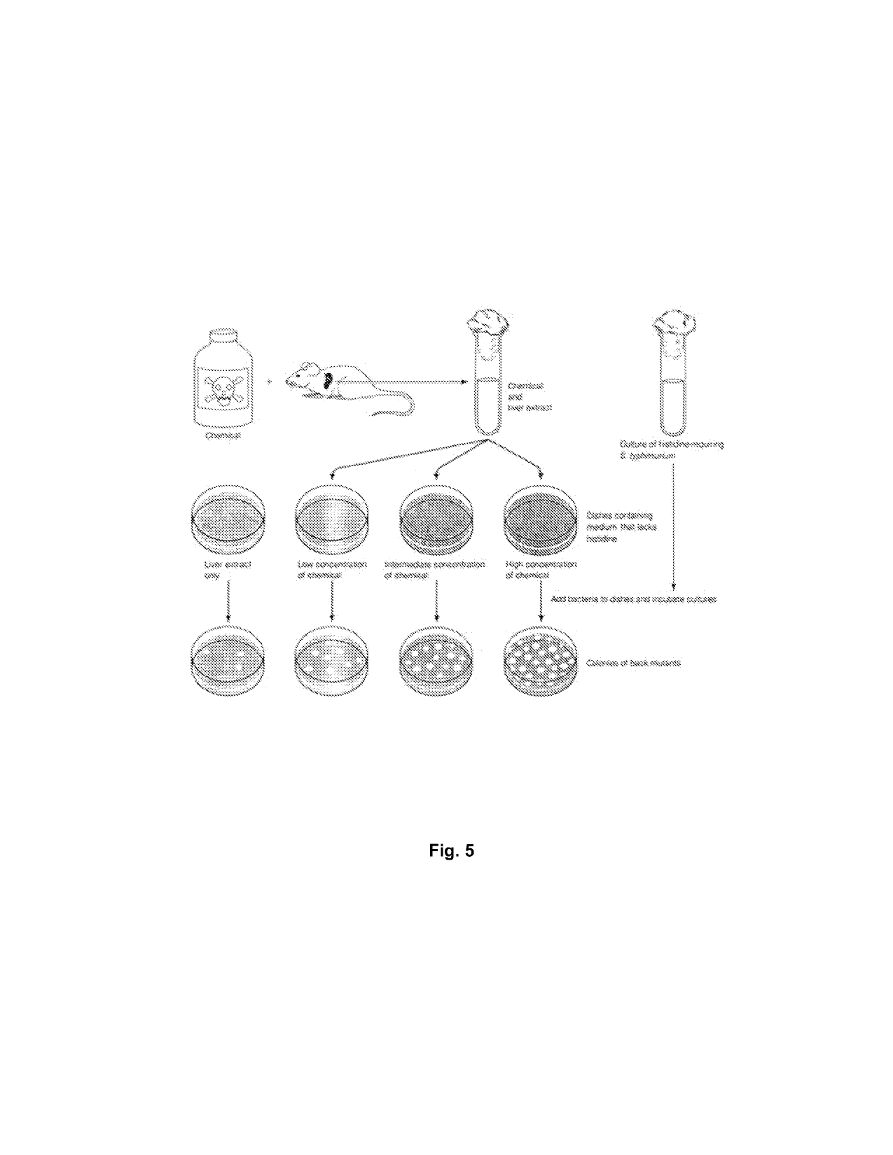

The Amest Test (FIG. 5) is based in the use of bacterial strains used to detect mutations in vitro (inheritable changes) in the DNA, 5 bacterial strains are used that allow the detection of the types of genome damage (changing a nucleotide base for another, shifting the reading frame) of the genes involved in histidine synthesis. Also, it was used the extract of rat liver microsomal (in humans we have the same enzymes that metabolize the products that come into our digestive system), with which, if an ingested component or food eaten by humans is metabolized and transformed into mutagenic/carcinogenic, this system can detect it in this Test. The Test with and without the liver microsomal fraction, should be repeated 3 times and in triplicate, for each dose of the studied sample, with positive and negative controls. This ensures properly updated designs studies, its reporting and on time studies and the interpretation in the context of the safety product results and the compliance with international regulations.

The Induc Test is the evaluation of the E. Coli K12 for the detection of potentially carcinogenic chemicals. Basically, the test consists in that this strain is infected with a phago in dormant or non-active phase. When a compound or certain environmental conditions or physical agents directly affect the DNA the REC system of the infected bacteria is activated to repare the damaged caused in the DNA. At that moment, the REC system “takes out” the virus repressor integrated to the DNA, stimulating this way the freeing and the viral multiplication, thus having a reduced number of bacteriae colonies.

The fundamental difference between the Ames Test and the Induc Test, is that in the first one it is possible to detect only damage in any of the genes that participate in a OPERON HIS while in the second one, it is possible to detect damage in any part of the bacterial genome.

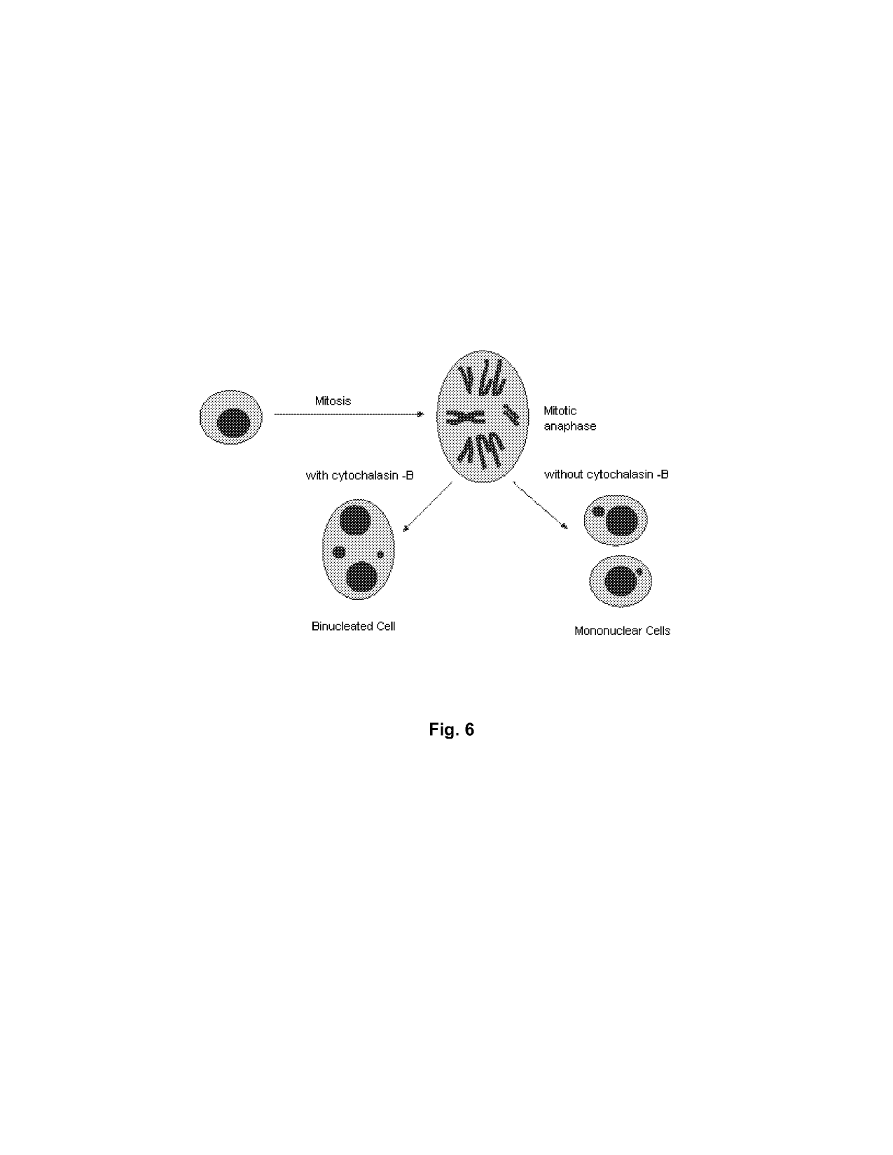

The micronucleus Test is the method of estimation of chromosomic damage in eukaryotes in a tissue that is constantly being divided such as the hematopoietic. During the cell division, the genetic material (DNA) contained in the cell nucleus is equally replicated and divided, resulting in two daughter cells, identical. This process can be erroneously made due to errors produced during the replication and subsequent DNA division, chromosome breakage and the effect of radiation and genotoxic agents, resulting in chromosomal loss and making the distribution of genetic material non-equitable. When this happens, the genetic material that is derived and, therefore, is excluded and that does not incorporate itself correctly to the daughter cell nucleus, creates a new nucleus of a smaller size than the primary one, called “micronucleus” (MN), easily visible with the optical microscope. The obtained genetic material can derive from whole chromosomes or, more frequently, from acentric chromosomes fragments that are excluded from the new cell nucleus during the mitotic anaphase (FIGS. 5 and 6).

FIG. 5 shows the citocalastri-B blockade and the consequent production of binucleated cells without which it would be possible to observe mononuclear cells with chromosomal loss making it impossible to tell whether they are stem cells or daughter cells.

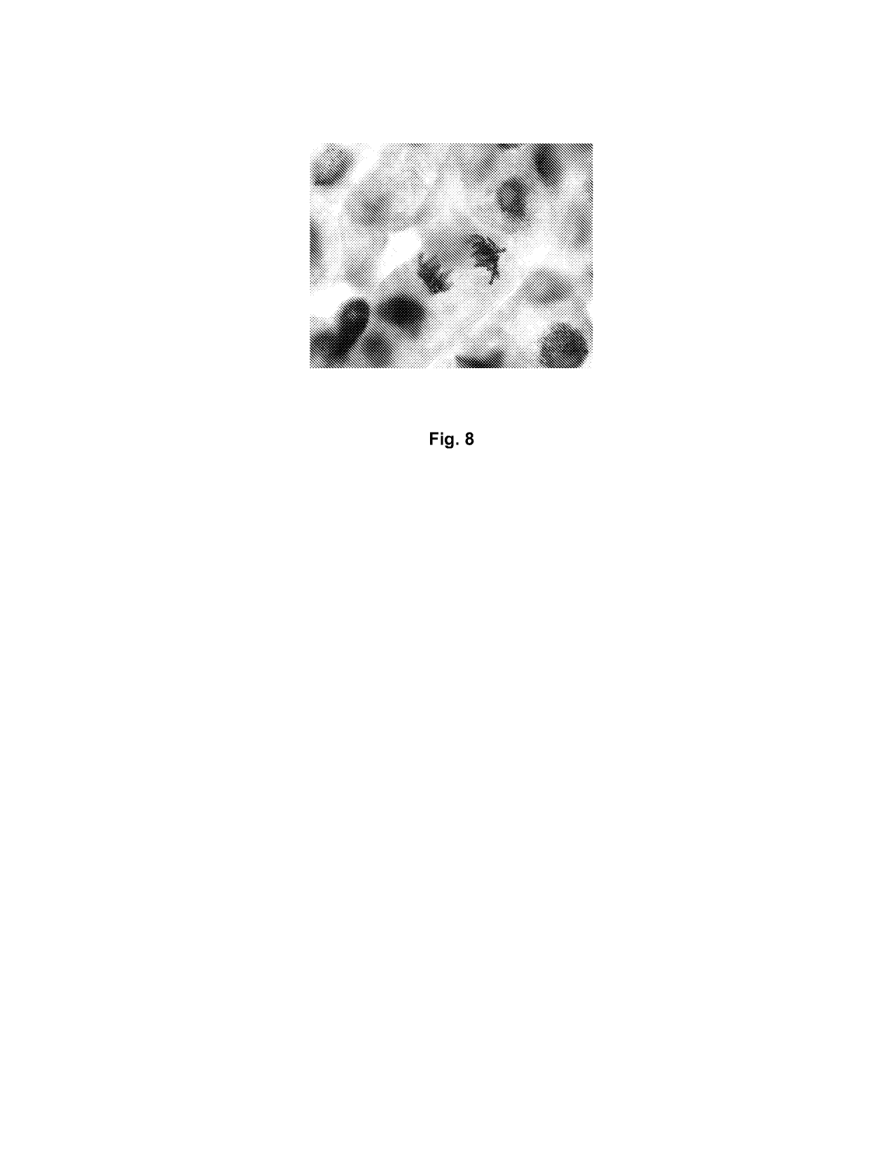

The anaphase aberration and sister chromatid exchange tests are produced by different mechanisms, but it is possible to observe them in a similar way, through light microscopy with Giemsa-stained tissues and/or fluorescence in peripheral blood (FIG. 8).

Sister chromatid exchanges are not lethal for the cell and, probably, do not present any relation with cytotoxicity. In addition, in themselves, they cannot be considered mutations because, in principle, they do not produce changes in the genetic information. But if is demonstrated that there is a damage in the chromosomal DNA in the studied compound and is valid the study under the hypothesis that certain cancers are a products of mutations accumulations.

However, it has been observed that the frequency of sisters cromatid exchanges increases when cells are exposed to mutagens and known carcinogens agents and also, in certain congenic diseases, i.e. Bloom Syndrome and Xeroderma pigmentosum.

At the same time, will be performed a in vivo analysis of the product. The effect teratogenic in pregnant female rats will be analyzed, and also the effect of promoting the development of tumours, using as a model the Sencar rats, an animal model sensitive to carcinogenesis. It was also analyzed the pharmaco-availability of the product, determining its intake and its excretion.

The molecule of interest has the following formulae:

R1 . . . C . . . R2

Wherein:

| | |
|---|---|
| R1 is a phenyl hydrazine PM | 108.14 |
| C is a Leucine | 131.17 |
| R2 is a methyl piperazine111 | 100.16 |
| | 339.69 |

Although embodiments of the invention have been shown and described, it is to be understood that various modifications, substitutions, and rearrangements of parts, components, and/or process (method) steps, as well as other uses of the molecule can be made by those skilled in the art without departing from the novel spirit and scope of this invention.

1. A molecule with following formula:
R1 R2 . . . C . . .
wherein:

| | |
|---|---|
| R1 is a phenyl hydrazine PM | 108.14 |
| C is a Leucine | 131.17 |
| R2 is a methyl piperazina 111 | 100.16 |
| | 339.69. |

2. A process comprising inhibiting Streptococcus mutans bacteria responsible for tooth decay by applying the molecule described in claim 1 to teeth.

3. A process comprising use of the molecule according to claim 1 for preparing different products used in oral hygiene.

4. A process according to claim 3 wherein the products remain under 60 seconds in the mouth.

5. A method of extraction of the molecule of claim 1, comprising the steps of:
selecting a most suitable organic medium to resuspend in an organic solvent suitable to maintain the physical-chemical characteristics (LogP, polarity index, dielectric constant, etc.) similar to a solvent used in the initial extraction, in order to corroborate effectiveness of the original extract;
performing thin layer chromatogaphy (TLC), using solvents with suitable physicochemical characteristics (especially polarity index, polarity and dielectric constant which are fundamental in liquid chromatography) for optimal separation of molecules present in the extract;
performing column chromatography (CCC) under substantially the same conditions in which was made for the thin layer chromatography (TLC) to obtain fractions with higher amounts of each of the separate molecules;
analyzing the fractions by high performance liquid chromatography (HPLC) to confirm the purity of the fractions obtained above; and
making biological evaluations with each of the fractions to identify the molecule responsible for inhibitory activity of S. mutants.
